(19) United States
(12) Patent Application Publication
(10) Pub. No.: US 2026/0221218 A1
(43) Pub. Date: Jul. 30, 2026

(54) COMPUTATIONAL SYNTHESIS AND VALIDATION OF APTAMERS

(71) Applicant: 4233999 Canada Inc., Westmount (CA)

(72) Inventors: Najeeb Ashraf KHALID, Westmount (CA); Ilias HURLEY, Montreal (CA); Adnan KHALID, Laval (CA)

(21) Appl. No.: 19/040,558

(22) Filed: Jan. 29, 2025

Related U.S. Application Data
(60) Provisional application No. 63/750,034, filed on Jan. 27, 2025.

(51) Int. Cl. G16B 15/30; G16B 5/00

(52) U.S. Cl. CPC G16B 15/30; C12N 2310/16

(57) ABSTRACT

For an intended target molecule, a candidate aptamer is designed using a plurality of short nucleotide sequences consecutively connected into an open-ended chain using connectors provided by double-stranded or single-stranded DNA structures. An interaction between 3D structures of the candidate aptamer and the target molecule using a docking algorithm predicts a docking pose in a simulated detection medium, which may be provided by liquid medium. The binding strength between the structure and target molecule is estimated while allowing the aptamer to conform to the target. If the binding strength is suitable, a morphological change of the aptamer upon binding is used to determine whether the aptamer is suitable for synthesizing.

### TECHNICAL FIELD

The present application relates to computational synthesis of aptamers for use in drug conjugation, or detection of pathogens, or measurement of biomarkers, and to the selection or design of aptamers.

### BACKGROUND

Aptamers are short single-stranded RNA or DNA molecules that can selectively bind to specific target molecules with high affinity, including proteins, peptides, small molecules, carbohydrates, live cells, etc. Aptamers assume a variety of shapes due to their tendency to form helical structures and single-stranded loops. Aptamer-based biosensors have been developed for use with a wide variety of sensing technique, such as electrochemical, optical, and mass-sensitive analytical techniques. Aptamers exhibit many advantages as recognition elements in biosensing when compared to traditional antibodies. They are small, chemically stable and cost effective. More importantly, aptamers offer remarkable flexibility and convenience in the design of their structures, which has led to novel biosensors that have exhibited high sensitivity and selectivity. They are synthetic antibodies and can be applied to replace antibody drug conjugates.

Target recognition and binding of aptamers to their targets are typically shape-dependent and predominantly driven by electrostatic interactions between an aptamer and a target molecule. Aptamer structure may be defined to ensure high affinity with its selected target molecule and no interaction with other molecules, which may be present in the expected environment, i.e., expected detection medium. This feature of aptamer molecules makes them indispensable and extremely efficient when it comes to the task of, for example, pathogen detection. However, creating an aptamer, allowing for efficient and accurate target detection may be challenging.

It is known in the art that, for example, SELEX (Systematic Evolution of Ligands by Exponential Enrichment) process, which involves generating a large, randomized pool of nucleic acid sequences and then isolating those sequences that bind to a selected target molecule, while discarding non-binders, enriches the pool for high-affinity candidates over multiple cycles. However, SELEX process, while powerful in principle, may be inherently limited by its reliance on iterative selection cycles and may be considered as a trial-and-error process. Apart from the SELEX database, there exist additional databases comprising catalogs of nucleotide sequences for well-studied target molecules. However, the structures of aptamers sourced from databases may not be optimized for specific application and, therefore, may need to be altered to enhance their binding to a specific target molecule. Since aptamers highly depend on their three-dimensional conformation for target binding, a refined process of designing their initial one-dimensional or primary structure is highly required to overcome the above-mentioned limitations. In other words, there exists a need for a method allowing to design an aptamer starting from a one-dimensional sequence of nucleic acids, which may be considered as potential candidate aptamer exhibiting desired binding affinity to an intended target molecule. In other words, there exists a need for a method allowing to synthesize an aptamer starting from a known target molecule.

### SUMMARY

Since experimentally selecting a suitable aptamer for a specific target molecule is an extremely challenging task, the Applicant has invented a process allowing one, using computational methods, to synthesize an aptamer for further binding to a selected intended target molecule, for example, as a detection element in a diagnostic test or an aptamer carrier that can be bound to an active compound of a drug for drug delivery.

More specifically, the Applicant has invented a method for computationally synthesizing an aptamer using a plurality of short nucleotide sequences, which may bind to a selected intended target molecule. First, a set containing a plurality of short nucleotide sequences, wherein each short nucleotide sequence may comprise at least two nucleotides, may be defined. To assess the binding between at least one short nucleotide sequence of the set and at least one binding location of an intended target molecule, a plurality of binding poses may be determined for each short nucleotide sequence of the set. Furthermore, sequences corresponding to at least two binding poses that spatially do not overlap and whose estimated binding strengths are acceptable, may be selected as candidate sequences to form an aptamer. Furthermore, those candidate nucleotide sequences may be linked together using corresponding connectors.

It may be appreciated that connectors may be defined to be structurally simple, i.e., being provided by backbones of nucleic acids or at least one nucleotide of a single-stranded DNA, flexible, i.e., being provided by single-stranded DNA, or rigid, i.e., being provided by double-stranded DNA. Moreover, spacers that are provided by single-stranded DNA structures may further be used to link a short nucleotide sequence and a connector. Additionally, for an aptamer containing at least one rigid connector, a single-stranded DNA may be connected between the less constrained end of an aptamer and a single strand of the at least one rigid connector, in order to form a loop, which may allow to transform an aptamer into a single-stranded structure. It may be appreciated be a person skilled in the art that once the structure of an aptamer has been computationally synthesized, it may be further synthesized in vitro.

The structure of computationally synthesized aptamer may be further validated using the following steps. Validation of the 3D structure of an aptamer may allow to confirm that the structure of computationally synthesized aptamer is accurate, relevant and reliable. To proceed with validation process, a one-dimensional (1D) or primary structure of an aptamer, which may be provided by a sequence of nucleotides, may be extracted from a computationally synthesized aptamer. This primary structure can then be used to predict a two-dimensional (2D) or secondary structure of an aptamer, and, furthermore, a three-dimensional (3D) or tertiary structure of an aptamer. The predicted 3D structure of an aptamer may then be used to validate the 3D structure of a computationally synthesized aptamer or provide an improved and more reliable conformation of a computationally synthesized aptamer. This may help to minimize artifacts that may have been introduced into the structure of computationally synthesized aptamer. Moreover, the validation process may allow to reduce uncertainties in further simulation of aptamer binding to an intended target molecule, i.e., it may help to ensure that the structure of computationally synthesized aptamer has the same or a very similar conformation as the one that has been predicted using the validation process.

Furthermore, aptamer's binding to an intended target molecule may be also validated. For that, an equilibrated aptamer structure and equilibrated intended target molecule may be placed into a vacuum, and a docking algorithm may be used to allow an aptamer to conform to an intended target molecule and, therefore, predict at least one docking pose. Usually, this process may involve predicting several docking poses, placing an aptamer-target complex in a water box, simulating molecular dynamics, and evaluating acceptable aptamer-target poses based on binding strength and stability assessment of an aptamer-target complex. Furthermore, when assessing binding of a candidate aptamer to an intended target molecule, unintended target molecules, which may be found in the potential test environment, may be equilibrated in an expected detection medium, i.e., a simulated liquid medium, and docked to further assess cross-reactivity between unintended target molecules and an aptamer.

### BRIEF DESCRIPTION OF THE DRAWINGS

The accompanying drawings, which are incorporated herein and form a part of the specification, illustrate embodiments of the disclosed invention and, together with the description, serve to explain the principles of the invention. In the drawings:

FIG. 1 schematically illustrates the process of computationally synthesizing the structure of a candidate aptamer by connecting short nucleotide sequences using corresponding connectors;

FIG. 2A schematically illustrates two neighboring short nucleotide sequences SAS1 and SAS2, which may be located in proximity to the surface of an intended target molecule and may be linked by a simple connector (see connector C0), for example, provided by the sugar-phosphate backbone of a nucleic acid; wherein SAS1 and SAS2, for the purpose of illustration, are provided by sequences comprising four nucleotides “TAGC” and “TCGA”, respectively;

FIG. 2B schematically illustrates short nucleotide sequences SAS1 and SAS2, which may be connected using a simple flexible connector (see connector C0′), which may be composed of one nucleotide, e.g., a nucleotide N1;

FIG. 2C schematically illustrates short nucleotide sequences SAS1 and SAS2, which may be connected using a simple flexible connector (see connector C0′), which may be composed of two nucleotides, e.g., nucleotides N1 and N2;

FIG. 2D schematically illustrates defining a location for placing a rigid connector with a junction (see connector C1 in FIG. 2F), which may be provided by double-stranded DNA and may allow to connect two proximal short nucleotide sequences, e.g., SAS1 and SAS2;

FIG. 2E schematically illustrates measuring the principal angle α, which may correspond to an angle at the junction of the potential rigid connector;

FIG. 2F schematically illustrates selecting a rigid connector with a junction (e.g., a connector C1), wherein the angle at the junction provided by the value of α;

FIG. 2G schematically illustrates transformation of a selected rigid connector with a junction (e.g., using rotation and/or translation operations), in order to extend the arms of that connector, allowing it to potentially connect to SAS1 and SAS2;

FIG. 2H schematically illustrates connecting a selected rigid connector with a junction to the oxygen atoms of the terminal nucleotides of SAS1 and SAS2 using spacers (e.g., see spacers D1 and D2), which may be proved by a single-stranded DNA;

FIG. 21 schematically illustrates defining a location for placing a rigid connector without a junction (see connector C2 in FIG. 21), which may be provided by the structure of a double-stranded DNA, allowing to connect two proximal SASs, i.e., SAS1 and SAS2;

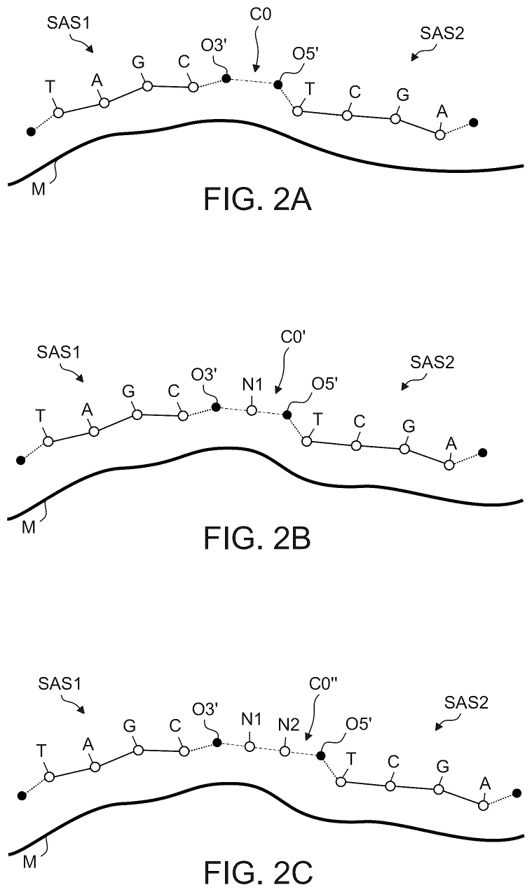

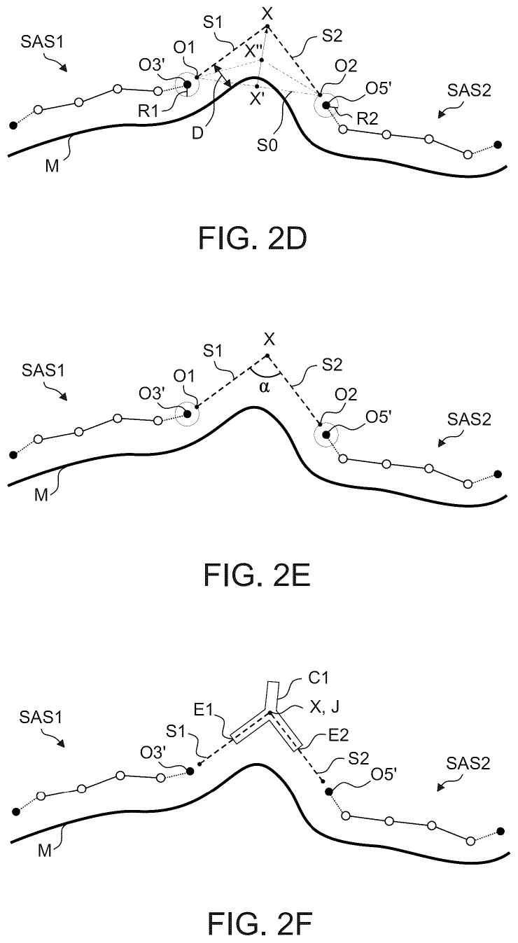

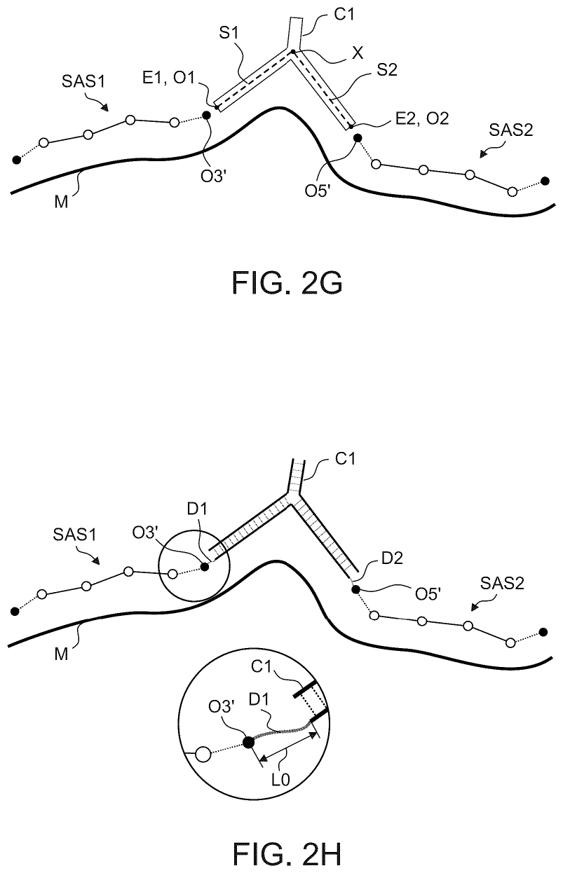

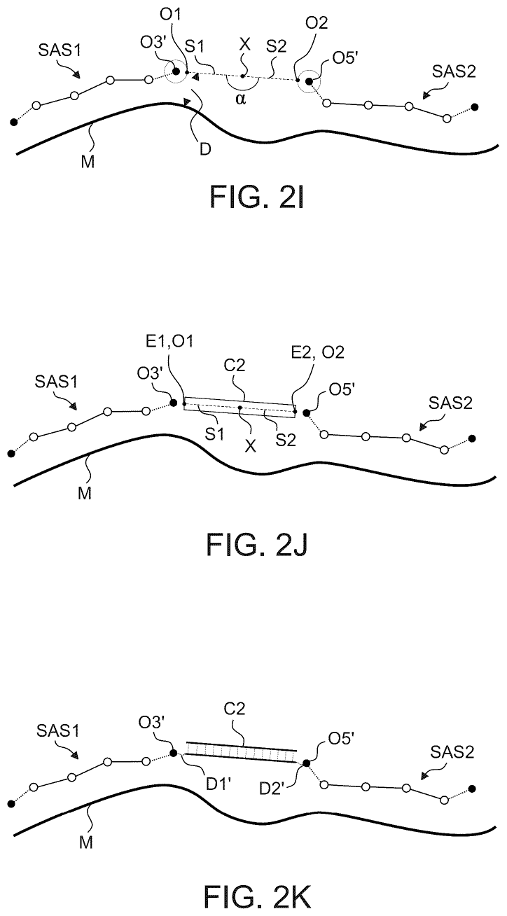

FIG. 2J schematically illustrates selecting a rigid connector without a junction (e.g., a connector C2) and applying transformation operation (e.g., rotation, translation, or both), in order to extend the arms of that connector, allowing it to potentially connect to SAS1 and SAS2;

FIG. 2K schematically illustrates connecting a rigid connector without a junction to the oxygen atoms of the terminal nucleotides of SAS1 and SAS2 using spacers (e.g., see spacers D1′ and D2′), which may be proved by a single-stranded DNA;

FIG. 3 schematically illustrates a global SAS assembly in the presence of the surface of an intended target molecule. In this example, four SASs are linked into open-ended chain, which may allow them to conform to the surface (see smoothed surface M) of a portion on an intended target molecule. It may be appreciated that three connectors (i.e., a connector with a junction C1′, a simple link C2′, and a connector without a junction C3′) may be used to connect respective SASs. Furthermore, spacers, which may be provided by single-stranded DNA (i.e., spacers SP1, SP2 and SP3), may be used to transform the global SAS assembly into a single, linear DNA sequence;

FIG. 4 schematically illustrates the process on aptamer structure validation.

FIG. 5 schematically illustrates the process of docking an aptamer to a target molecule;

FIG. 6 schematically illustrates the process of assessing the binding of an aptamer to a target molecule;

FIG. 7 schematically illustrates the process of assessing cross-reactivity between an aptamer and other molecules, which may be present in a solution;

FIG. 8 illustrates a perspective view of a 3D aptamer structure;

FIGS. 9A and 9B illustrate a perspective view of the aptamer structure placed into a simulated liquid medium (i.e., a solution provided within a 3D bounded region, such as, for example, a water box) before and after energy optimization, respectively;

FIGS. 10A and 10B illustrate a perspective view of the 3D structure of a target molecule placed into a simulated liquid medium (i.e., a solution) before and after energy optimization, respectively;

FIGS. 11A to 11C illustrate possible docking configurations, i.e., docking poses, between an aptamer and its target molecule;

FIGS. 12A and 12B illustrate a perspective view of a 3D aptamer-target complex placed into a solution, i.e., a 3D aptamer-target complex-solution system before and after energy optimization, respectively; and

FIG. 13 illustrates a plot depicting the root-mean-square deviation (RMSD) for trajectory frames obtained during the binding assessment.

### DETAILED DESCRIPTION

It is explicitly stated that all features disclosed in the description and/or the claims are intended to be disclosed separately and independently from each other for the purpose of original disclosure as well as for the purpose of restricting the claimed invention independent of the composition of the features in the embodiments and/or the claims. It is explicitly stated that all value ranges or indications of groups of entities disclose every possible intermediate value or intermediate entity for the purpose of original disclosure as well as for the purpose of restricting the claimed invention, in particular as limits of value ranges.

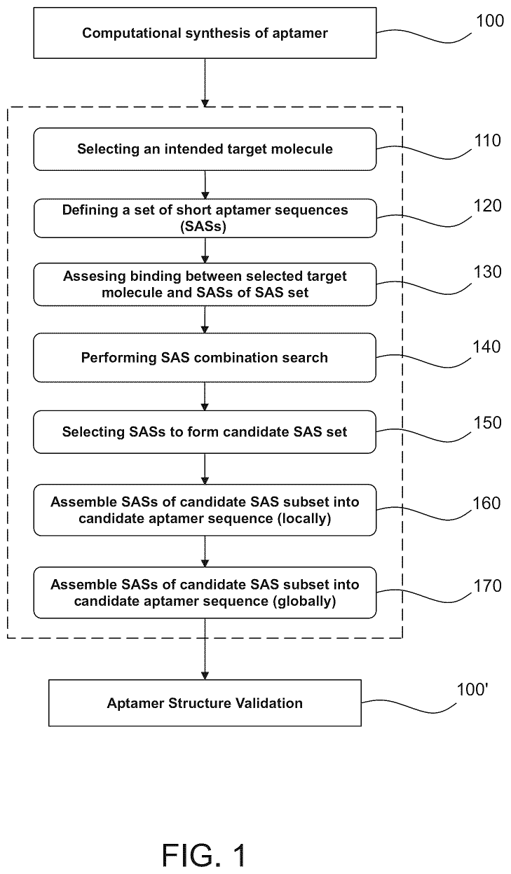

FIG. 1 schematically illustrates a method allowing for a computational synthesis of the 3D structure of a candidate aptamer. To begin, a user may first select an intended target molecule, which may be described using three-dimensional atomic coordinates, i.e., a ligand, which, for example, may be selected from Protein Data Bank (PDB) database or any other suitable source, as shown in step 110. Target molecules may typically be provided by biomolecular structures, such as proteins, multi-protein complexes, nucleic acids, and other molecules. It may be appreciated that an intended target molecule selected by a user (see, for example, FIG. 10A schematically illustrating a target molecule) may contain no inconsistencies in its structure, such as, for example, structural gaps and/or structural omissions, which may refer to missing or unresolved regions in the molecular structure provided, for example, in a PDB format and may arise due to experimental limitations or inherent flexibility in certain regions of the target molecule. Furthermore, all atom definitions may conform to the “Amber ff14SB” standard. In case, any structural inconsistencies have been identified, a user may use specialized tools (e.g., Rosetta, MODELLER, AlphaFold, etc.) to model unresolved regions, in order to reconstruct the molecular structure of an intended target molecule and ensure that modeled regions are realistic and validated against available data.

As further shown in FIG. 1, it may be appreciated that a set of short nucleotide sequences, which, in this example, may be composed of DNA nucleotides and may further be referred to as a set of short aptamer sequences (SASs) or, simply, a SAS set, (see step 120), may be pre-defined for further computations. It may be appreciated by a person skilled in the art that short nucleotide sequences may be provided by a sequence represented using four nucleotide bases, i.e., adenine (A), cytosine (C), guanine (G), and thymine (T), all of which constitute the building blocks of DNA molecules. Alternatively, short sequences may be provided by RNA nucleotides, which correspond to DNA nucleotides, wherein uracil (U) is used in place of thymine (T). It may be appreciated by a person skilled in the art that, depending on the potential utility of a designed aptamer, DNA sequences may be converted to RNA format and vice-versa. Moreover, artificial DNA/RNA nucleotides, i.e., nucleotides with altered chemical structures not natively occurring in nucleic acids, may be also used to enhance the ability of SASs to bind to an intended target molecule.

In this embodiment, each SAS of a SAS set, for example, may be composed of four DNA nucleotides, and, therefore, may be represented by a four-character long sequence, which may be provided in a computer-readable format, such as, for example, TXT format. Furthermore, it may be appreciated that each SAS of a SAS set may be used as a building block of a candidate aptamer sequence. In this example, there are four choices for each position of DNA nucleotides in a four-character long sequence, which may result in 4⁴=256 SASs in total. However, those SASs that are entirely composed of adenine (A) and thymine (T), which are provided by the total of 2⁴=16 SASs, may be removed, as they may have a low probability of forming strong binding sites. Therefore, in this example, a SAS set may contain 240 SASs in total.

It may be appreciated by a person skilled in the art that, in alternative embodiments, longer or shorter SASs may be used. For instance, a SAS comprising only two or three nucleotides may be considered. Therefore, a respective SAS set may contain 4²−2²=12 or 4³−2³=56 SASs in total. A person skilled in the art may recognize that a smaller number of candidate SASs can be computationally assessed more quickly. However, they may also recognize that using shorter SASs may fail to model synergism between neighboring nucleotides, thus limiting their utility. Alternatively, a SAS comprising five or six nucleotides may also be considered. In this case, a respective SAS set may contain 4⁵−2⁵=992 or 4⁶−2⁶=4032 SASs in total. However, a person skilled in the art may recognize that increasing degrees of freedom by adding more nucleotides in a SAS would exponentially increase computational time. In addition, it may be appreciated by a person skilled in the art that known aptamers may rarely comprise long sequences of contiguous nucleotides that interact with an intended target molecule, and as such using long sequences may bias the results. Therefore, it may be appreciated that the Applicant has experimentally determined that a SAS set containing four-character long SASs may be preferred, as it may offer a better compromise between computational efficiency and quality and diversity of results.

Furthermore, each SAS may be associated with a unique identifier (ID). It may be appreciated that a unique ID, four-character long SASs, and the name of a PDB file containing the information about the 3D structure of an intended target molecule may be stored in a separate file on a computer-readable medium. Furthermore, the file may also contain information about the numbering of the atoms of the terminal hydroxide groups, which may be determined manually.

It may be appreciated by a person skilled in the art that each SAS may comprise a 5′ end and a 3′ end, which may be considered as the beginning and the end of a SAS, respectively. Furthermore, each SAS of a SAS set may be provided, for example, in the PDBQT format, where “Q” in PDBQT refers to “charges”, which may, for example, be provided by atomic partial charges and are essential for molecular docking simulations, and “T” in PDBQT refers to “torsions”, which may, for example, provide torsional degrees of freedom in a SAS, allowing for flexibility in the SAS's structure during its binding to an intended target molecule and enabling the exploration of different conformations. It may be appreciated that the PDBQT format may be tailored for programs like AutoDock, where both, detailed charge information and flexibility or torsions, may be important components for accurate simulation and scoring.

In order to assess the binding between the intended target molecule and a SAS of the SAS set (see step 130 in FIG. 1), a user may specify a potential binding site(s), for which an aptamer may be designed. It may be appreciated that a binding site, or, equivalently, a search region, may be provided by at least a portion of an intended target molecule. For example, a user may provide (x, y, z)-coordinates of the center point of a search region, which may contain a plurality of potential binding locations for SASs. For example, a binding site may be represented by a bounded 3D region with its dimensions specified by a user or may be automatically determined by a suitable software.

In this example, a binding site may be provided by a cubical box, wherein its edge length may be specified by a user, with the center of a binding site provided by the coordinates of the centroid of that cubical box. It may be appreciated that, in this example, the dimensions of the search region may be provided in Angstrom (Å). Also, it may be appreciated by a person skilled in the art that an entire intended target molecule may be considered as the potential binding site, i.e., a user may select, or a corresponding suitable software may automatically identify binding locations on the surface of an intended target molecule in order to assess further aptamer binding.

Furthermore, a user may specify a number of binding poses for each SAS, i.e., a number of spatial orientations to be generated for each SAS of the SAS set. This value is analogous to the resolution of the SAS binding search. In this example, at least 20 binding poses may be generated for each SAS of a SAS set to further allow for assessing the binding between each SAS pose and at least one binding location of an intended target molecule within the specified binding site. However, any other number of binding poses for each SAS may be considered. For instance, for a very large search region (side length >100 Å), at least 50 binding poses may be generated for each SAS. It may be appreciated that a SAS's binding pose, which has its terminal atoms bind to an intended target molecule, may be discarded, as binding of SAS's terminal regions to a target molecule may reduce the probability of other nucleotides comprised within the SAS to bind to a target molecule as well, therefore, preventing that SAS from being a potential candidate for an aptamer.

It may be appreciated by a person skilled in the art that binding poses may be generated for any number of SASs of a SAS set (e.g., for at least one SAS of a SAS set). For example, a user may generate a selected number of binding poses for all SASs comprised within a SAS set, or specify a number of SASs, for which binding poses may be generated, or even select specific SASs of interest (e.g., if there is a need to explore specific short nucleotide sequences) from a SAS set and explore their binding poses.

It may further be appreciated that, before generating SAS binding poses, an intended target molecule may be placed into a simulated liquid medium, for example, provided by a water box, for stabilizing the target molecule, which may improve simulation accuracy (see FIGS. 9A and 9B showing a target molecule in a simulated liquid medium before and after relaxation, respectively).

As further schematically illustrated in FIG. 1, once binding poses have been identified, a SAS combination search (see step 140) may be performed in order to find at least one subset of SASs of a SAS set, which contains SASs allowing to assemble a potential candidate aptamer. It may be appreciated that each SAS subset may only contain those SASs that exhibit a desired level of binding to an intended target molecule, i.e., demonstrate a binding energy below a determined threshold and have their terminal atoms in unbound state.

In this example, each possible SAS subset may contain five SASs; however, it may be appreciated that the smallest SAS subset may contain only two SASs, which may be preferred for small target molecules. It may also be appreciated by a person skilled in the art that a SAS subset comprising more than five SASs (e.g., containing eight or ten SASs) may also be used as a candidate SAS subset for forming aptamer's structure. For example, large SAS subsets, i.e., subsets containing more than five SASs, may be pertinent in case of large target molecules, or those target molecules that have a small amount of high-quality binding sites, and therefore, may require an aptamer to contain more nucleotides within its structure, potentially allowing to increase its chances to bind to an intended target molecule.

In one embodiment, the members of SAS subset may be provided by distinct SASs. However, it may be appreciated that, in an alternative embodiment, the members of a SAS subset may be provided by the same SAS having different spatial orientations, i.e., different binding poses (see, for example, a subset of four SASs linked into an open-ended chain, which is schematically illustrated in FIG. 3).

Furthermore, SASs forming a SAS subset may not overlap in space, i.e., the 3D bounded regions occupied by each SAS may be located within a certain distance from each other, wherein the distance threshold may be specified by a user. In this example, two SASs may be considered as overlapping, if the distance between their corresponding bounded 3D regions, i.e., the approximated 3D regions tightly enclosing corresponding SASs, is less than 1 Å. It may be appreciated that a distance threshold may vary, and may, for example, be provided by 0 Å, 5 Å, or 10 Å, etc. It may be appreciated that if the condition for non-overlapping bounded 3D regions holds for each combination of SASs comprised within an examined SAS subset, the corresponding SAS subset may be considered as candidate subset for forming an aptamer (see step 150 in FIG. 1).

In this example, it may be appreciated that if no subsets of five non-overlapping SASs have been identified, the SAS combination search may be repeated in a similar way for all possible subsets of four SAS poses, three SAS poses, etc. It may further be appreciated that SASs forming a SAS subset may also have the lowest binding scores. In this case, the binding score is a numerical approximation of the binding strength between a SAS and an intended target molecule. Binding strength may be denoted by ΔG, i.e., the change in Gibbs free energy between the initial (unbound) and final (bound) states. Since a negative ΔG denotes a spontaneous process, a person skilled in the art may recognize that finding the lowest possible binding strength or binding score for each SAS of an examined SAS subset is desirable. The total binding score for each SAS subset may be obtained by adding together the binding scores of each SAS comprised within an examined SAS subset. The SAS subset with the lowest total binding score may then be selected as a candidate SAS set for creating an aptamer and may serve as input for local aptamer assembly.

As further schematically illustrated in FIG. 1, in order to locally assemble an aptamer, the terminal atoms of pairs of spatially neighboring SASs constituting a candidate SAS set, may be connected to form an open-ended chain. Therefore, for every pair of neighboring SASs, the shortest path may be identified. To address this challenge, the surface of an intended target molecule may be provided by a surface polygon mesh approximation constructed based on the atomic coordinates of the atoms forming an intended target molecule. Moreover, the polygonal surface of the target molecule may be smoothed (see, for example, a 2D view of the smoothed surface M schematically illustrated by a smooth curve in FIGS. 2A and 3) using existing suitable algorithms for 3D surface smoothing, such as, for example, Laplacian smoothing, Taubin smoothing, Gaussian smoothing, etc.

Coming back to step 160 of FIG. 1, all neighboring SASs within a candidate SAS set may be connected pairwise to form a single open DNA strand. It may further be appreciated by a person skilled in the art that, in order to join any two SASs, a terminal nucleotide of a SAS corresponding to the 3′ end of one SAS may be connected to a terminal nucleotide of the 5′ end of another SAS (see a pair of “TAGC” and “TCGA” SASs in FIGS. 2A to 2C, which are selected solely for the purpose of illustration). The corresponding terminal nucleotides may then be considered as a potential pair of nucleotides to be connected to form the aptamer's structure. It may further be appreciated that the distance between terminal nucleotides may be measured for each potential pair of neighboring SASs comprised within a candidate SAS set, and only those two SASs that have the shortest distance between their terminal nucleotides may be connected. However, it may be appreciated that any other suitable techniques for connecting pairs of SASs within a candidate SAS set that are known in the art may be used to assemble an aptamer. Therefore, it may be appreciated that, for the K number of SASs of a candidate SAS set, K-1 connection paths may be inserted between each pair of SASs, in such a way that SASs may form one open DNA strand. For example, if a selected SAS subset contains four SASs, three connection paths may be estimated to connect those SASs (see, for example, in FIG. 3, four SASs of a candidate SAS subset connected together using three connectors).

It may further be appreciated that different types of connection paths may be used depending on the distance between two terminal nucleotides corresponding to different SASs within a selected SAS subset. In order to maximize the reduction of conformal entropy (i.e., a measure of the number of ways an aptamer can arrange itself spatially due to its rotational, vibrational, and other internal degrees of freedom) of the resulting aptamer structure, it may be appreciated that the connection paths may be provided by rigid molecular structures, which may further be referred to as rigid connectors. However, the structural configuration of a rigid connector may be larger and more complex comparatively to molecular structures of SASs, which may prove challenging to use such connectors for connecting a pair of SASs, which, for example, are located at a distance that is smaller than their lengths, due to the risk of steric hindrance. Therefore, in certain cases, it may be preferable to use flexible connectors instead.

In this embodiment, flexible connectors may be constructed from a single-stranded DNA, as they may be easier to implement than other polymers since the structure of each SAS comprised within a candidate SAS set may be provided by DNA nucleotides. In an alternative embodiment, other flexible connectors, such as, for example, synthetic polymers (i.e., artificially synthesized materials composed of long chains of repeating monomers, e.g., polyethylene glycols), may be used. However, it may be appreciated that, in the case of artificially synthesized connectors, post-synthesis conjugation may be required in order to manufacture the resulting aptamer.

In this embodiment, connecting SASs comprised within a candidate SAS set using small simple connectors may be considered as a part of local aptamer assembly (see step 160 in FIG. 1). It may be appreciated by a person skilled in the art that if the distance between terminal nucleotides of the corresponding two SASs is more than 1 Å but less than 2 Å, such distance may be equivalent to a distance between any two consecutive nucleotides in a SAS. Therefore, such terminal nucleotides may be simply linked, i.e., may be joined without a spacer (e.g., a structure provided by a single-stranded DNA), for example, by their sugar-phosphate backbone (for example, see the connector C0 linking terminal nucleotides of SAS1 and SAS2 schematically illustrated in FIG. 2A, which, for the purpose of illustration, may be provided by C nucleotide of “TAGC” sequence representing SAS1 and T nucleotide of “TCGA” sequence representing SAS2).

Furthermore, in this example, if the distance between a pair of proximal terminal nucleotides of two corresponding SASs is more than 2 Å but less than 12 Å, such pair of SASs may be joined by using a flexible connector composed, for example, of one or two nucleotides (see, for example, FIGS. 2B and 2C schematically illustrating a connector C0′ composed of one nucleotide N1 and a connector C0″ composed of two nucleotides N1 and N2, respectively). It may be appreciated that a connector comprising more than two nucleotides may also be used, however, such flexible connectors increase entropy and therefore may not be preferred. As illustrated in FIG. 2B, nucleotide N1 of a flexible connector C0′, for example, may be provided by a single thymine (T) nucleotide. Although any other nucleotides (e.g., A, C, G, or artificial nucleotides) may be used instead, thymine (T) nucleotides may be preferable due to their size and low binding strength, which may make them less likely to interfere with the structure of a SAS.

Once the local aptamer assembly has been performed, the global SAS assembly (see step 170 in FIG. 1), i.e., the aptamer assembly using structurally large and complex connectors, may be performed. In this example, it may be appreciated that the global aptamer assembly may be considered, when the distance between two proximal terminal nucleotides of any two SASs comprised within the selected SAS set is more than 12 Å. Furthermore, it may be appreciated by a person skilled in the art that rigid connectors may be provided by double-stranded DNA, which may have a radius of about 10 Å; however, any other types of rigid connectors that are known in the art may be used instead, if they are compatible with SASs of a candidate SAS set. Using double-stranded DNA as a rigid connector may provide compatibility and easier integrability with SAS structure, while offering a rigid connection between a pair of SASs, i.e., a connection which may not be significantly altered during binding of the aptamer to an intended target molecule.

The global SAS assembly may take as an input the 3D structure of an intended target molecule, its smoothed surface M, a candidate SAS set, wherein some of neighboring SASs may be locally connected during the local aptamer assembly (see step 160 of FIG. 1), and a set of connectors, which, in this example, may comprise double-stranded DNA structures that may be used to connect proximal SASs with their respective terminal nucleotides spaced by more than 12 Å. It may be appreciated that a set of DNA connectors may comprise DNA junctions with known inter-arm angles, which may be determined experimentally, for example, using FRET, NMR, AFM, or any other suitable techniques known in the art that are capable of estimating nucleotide positions in 3D space.

The process of global SAS assembly (see step 170 in FIG. 1) is schematically illustrated in FIGS. 2D to 3 and may comprise the following steps. As shown in FIG. 2D, for each pair of neighboring SASs comprised within a candidate SAS set (in this example, see SAS1 and SAS2, which, for the purpose of illustration, are provided by short aptamer sequences comprising four nucleotides schematically illustrated by white nodes), the coordinates of the oxygen atoms linked to the terminal nucleotides may be determined (see the oxygen atoms O3′ and O5′ linked to the terminal nucleotide corresponding to 3′ end of SAS1 and the terminal nucleotide corresponding to 5′ end of SAS2, respectively). Furthermore, a sphere of radius R1 may be centered at a point provided by the coordinates of the oxygen atom O3′, and a sphere of radius R2 may be centered at a point provided by the coordinates of the oxygen atom O5′. It may be appreciated that R1 and R2 may be provided by the same value (in this example, R1 and R2 may have a value of 4A). Following that, the points O1 and O2, which are found on the surface of the spheres centered at O3′ and O5′, respectively, may be automatically selected. It may be appreciated that the points O1 and O2 may be considered as being equivalent to the end points of the rigid connector being determined.

Furthermore, the coordinates of a point X, which may be connected to the points O1 and O2 using segments S1 and S2, respectively, may be determined. It may be appreciated that the coordinates of the point X may provide the coordinates of a junction point of the potential rigid connector, i.e., the central reference point of the connector. In order to estimate the coordinates of X, an existing software and tools for geometric computations, such as, for example, CGAL or any other suitable software, may be used. For example, the first approximation of the coordinates of point X may be provided by the point X′ found in the middle of the segment S0 connecting points O1 and O2. To continue approximation of the coordinates of X, the following constraints may be considered: first, the distance from any point lying on the segments S1 and S2 to the surface M of an intended target molecule (see distance D between S1 and M in FIG. 2D) may be greater than 10 Å (or any other suitable value depending on the type and components of the connector), which may protect potential connector from overlapping with the surface M, as the radius of double-helix DNA is about 10 Å; second, the total length of S1 and S2 may be required to meet a minimum value. Therefore, as schematically illustrated in FIG. 3D, the second approximation of X may be provided by the point X″, etc. After a certain number of approximations, the geometric computational software in use may efficiently determine final coordinates of X based on the provided constraints.

As further schematically illustrated in FIG. 2E, once the coordinates of the point X have been determined, it may be possible to calculate the angle between the segments S1 and S2, i.e., the principal angle α. It may be appreciated that the principal angle α may take any value from 1° to 180°. However, it may further be appreciated by a person skilled in the art that the values of the principal angle α may, for example, in practical setting, vary from about 20° to 180°, taking into account the diameter of DNA strands in a rigid connector (e.g., if the principal angle α is less than about 20°, the DNA strands in the rigid connector with a junction may overlap).

As further schematically illustrated in FIG. 2F, depending on the value of the principal angle α, a rigid connector having the same angle value may be chosen from the set of connectors (such as for example, connector C1, wherein the junction J of C1 may be aligned with point X). It may be appreciated that the set of connectors may, for example, comprise up to 180 connectors corresponding to each possible integer value of the principal angle α, however, only the connectors corresponding to the principal angle a from about 20° to 180° may be useful in a practical setting. It may be appreciated that E1 and E2 may denote the endpoints of the connector C1. Furthermore, connector C1 may be transformed using translation, rotation, or both, so that the arms of the connector may be aligned with the segments S1 and S2 (i.e., the segments S1 and S2 may provide imaginary central lines around which the double helix structure of DNA is wound). It may further be appreciated that a rigid connector may comprise more than one junction.

Moreover, as further schematically illustrated in FIG. 2G, the arms of the connector C1 may be extended by adding more nucleotide pairs to its structure, which, in turn, may allow the endpoints E1 and E2 of C1 to match the corresponding endpoints of the first and second segments S1 and S2, i.e., points O1 and O2, respectively. Moreover, a person skilled in the art may appreciate that the sequence of an extended arm may contain any combination of A-T and G-C base-pairs.

Furthermore, it may be appreciated by a person skilled in the art that directly connecting terminal nucleotides of the SASs to a connector provided by double-stranded DNA (see connector C1 in FIG. 2H schematically illustrated as double-stranded DNA connector with its connector arms extended along the segments S1 and S2 as shown in FIG. 2G) may be challenging, as the connector may interfere with the surface M of an intended target molecule or alter the behavior of terminal nucleotides. Therefore, as illustrated in FIG. 2H, a pair of single-stranded DNA spacers (e.g., spacers D1 and D2), may be inserted to connect each endpoint of the connector (e.g., points E1 and E2 of the connector C1) to the corresponding terminal oxygen atoms of SAS1 and SAS2 (such as, for example, single-stranded DNA spacer D1, which may be connected to the oxygen atom O3′ of the terminal nucleotide of SAS1, and single-stranded DNA spacer D2, which may be connected to the oxygen atom O5′ of the terminal nucleotide of SAS2). It may be appreciated that a spacer may span a gap of length L0. A person skilled in the art may appreciate that single-stranded DNA is quite flexible (having a persistence length on the order of Å), and as such a spacer may be longer than L0 (see a magnified view shown in FIG. 2H). Furthermore, it may be appreciated that for sufficiently small values of L0, such as, for example, 4A, a spacer may consist of a single nucleotide. A person skilled in the art may appreciate that a spacer may contain any of A, C, G, or T nucleotides, but may preferentially contain only T nucleotides due to their small size and low binding strength.

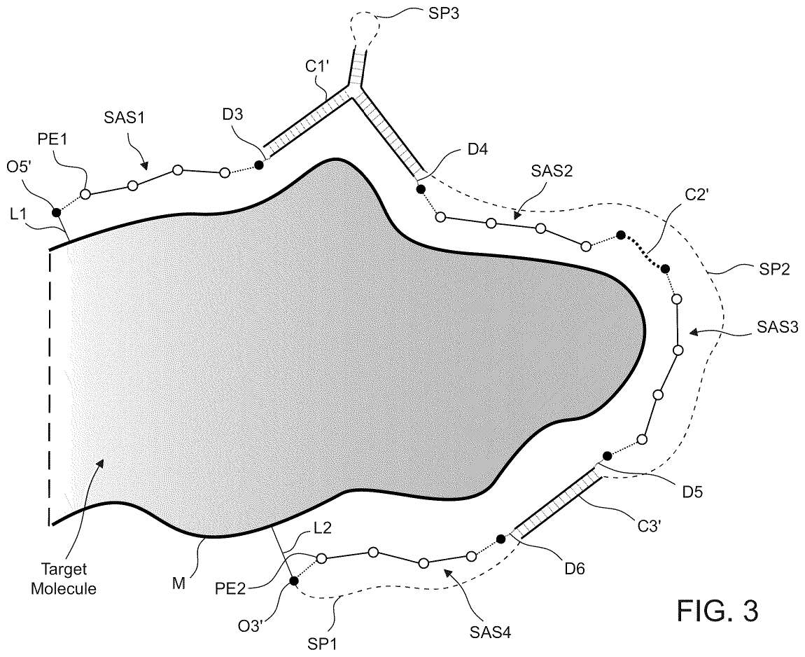

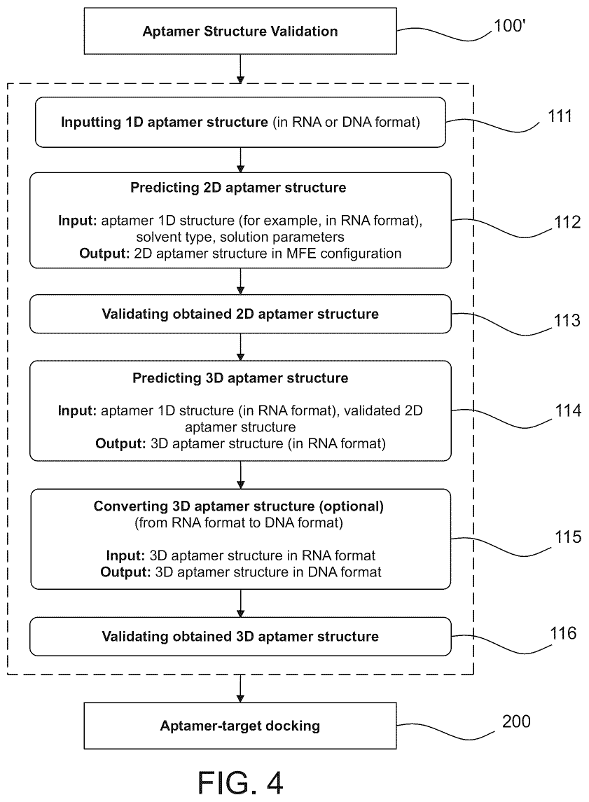

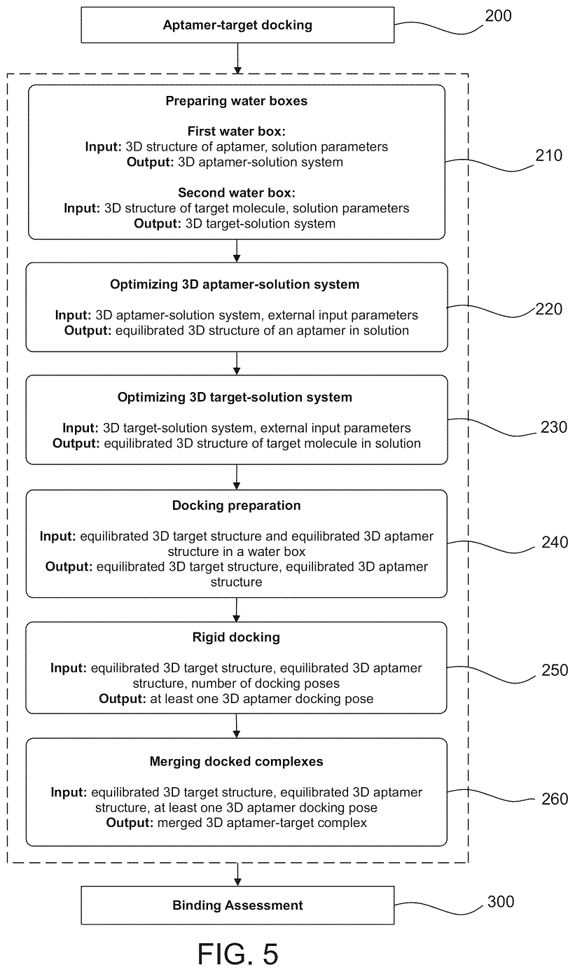

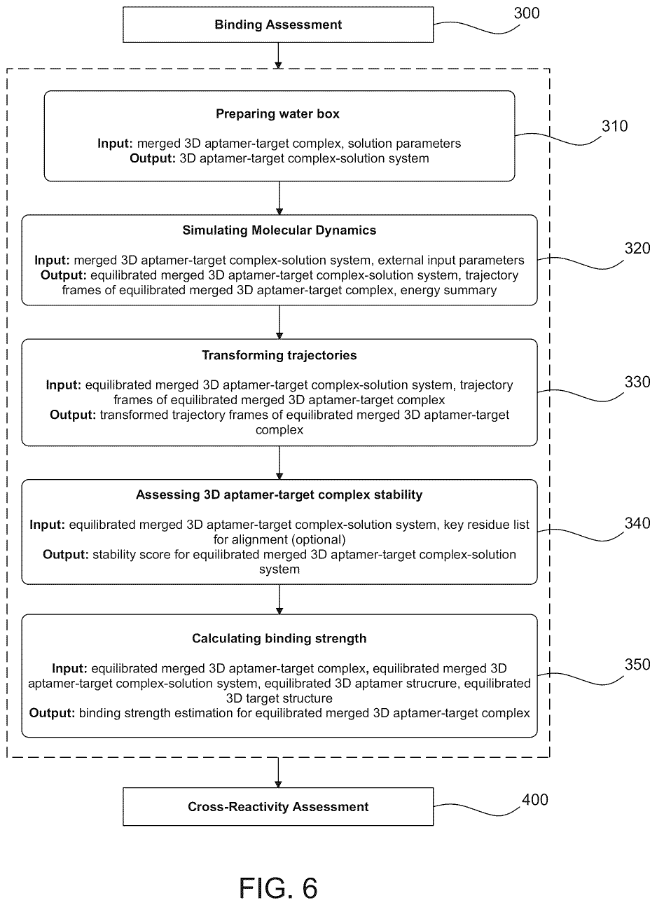

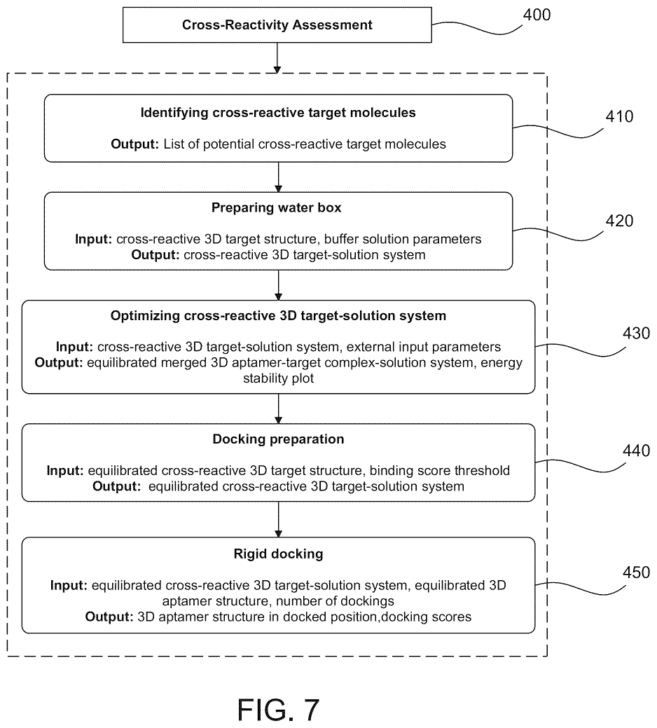

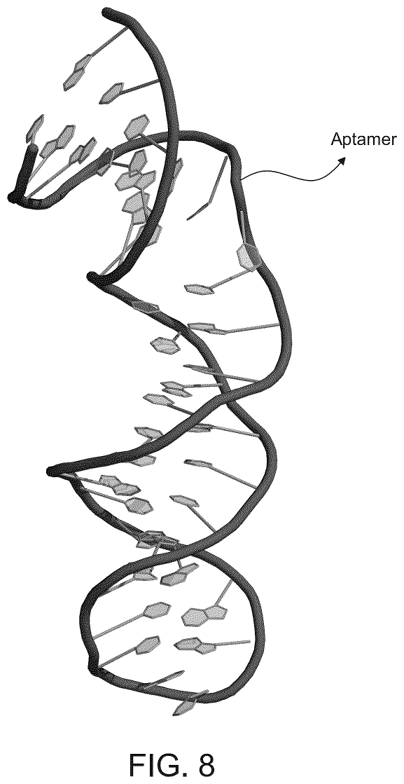

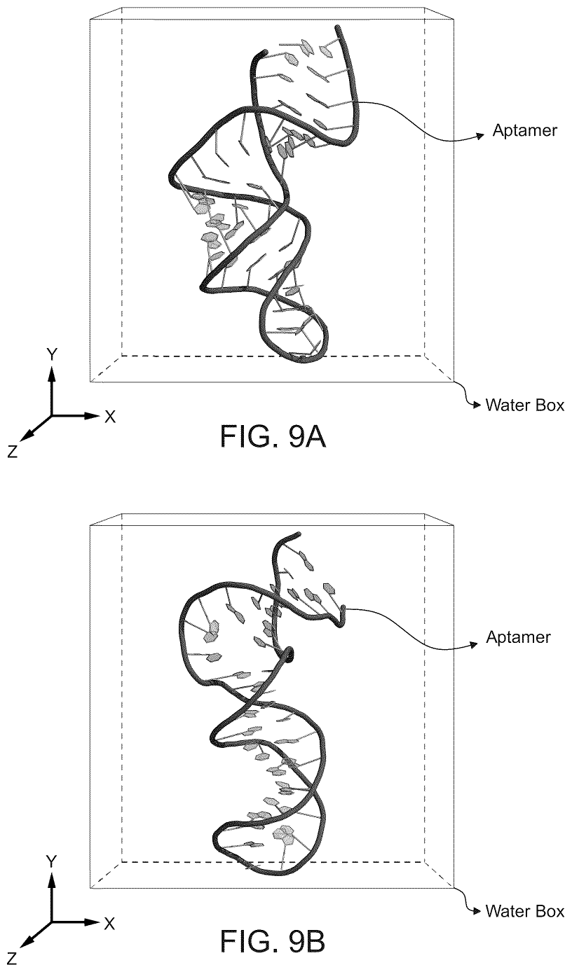

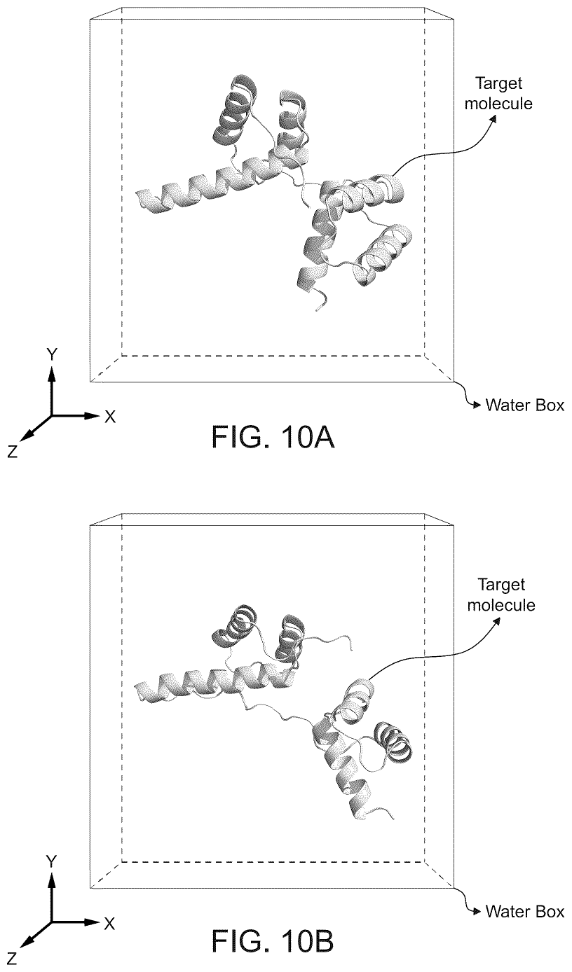

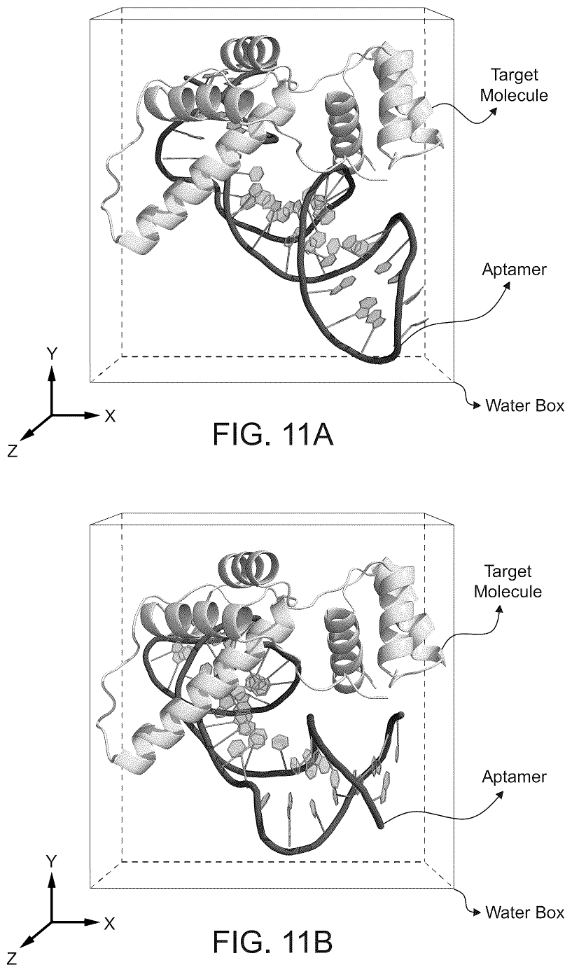

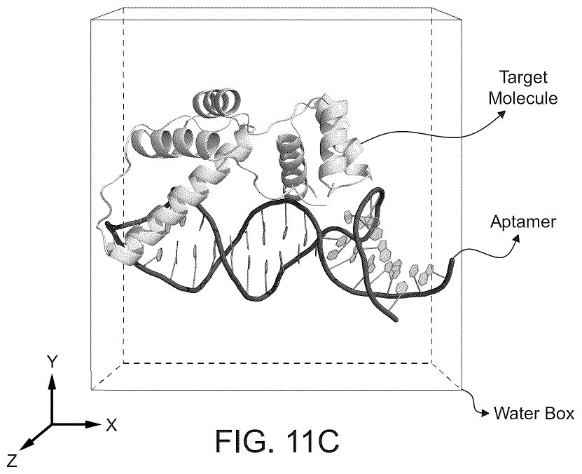

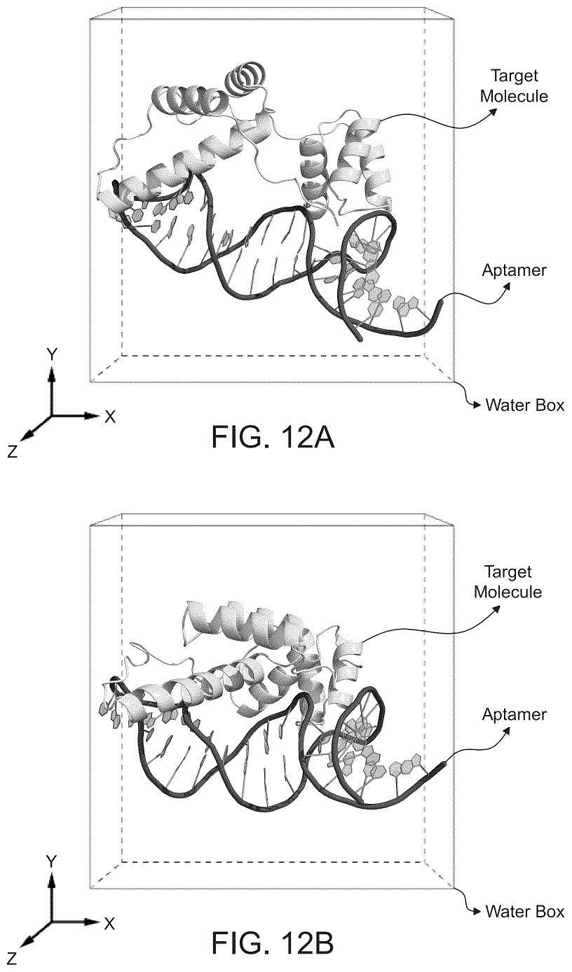

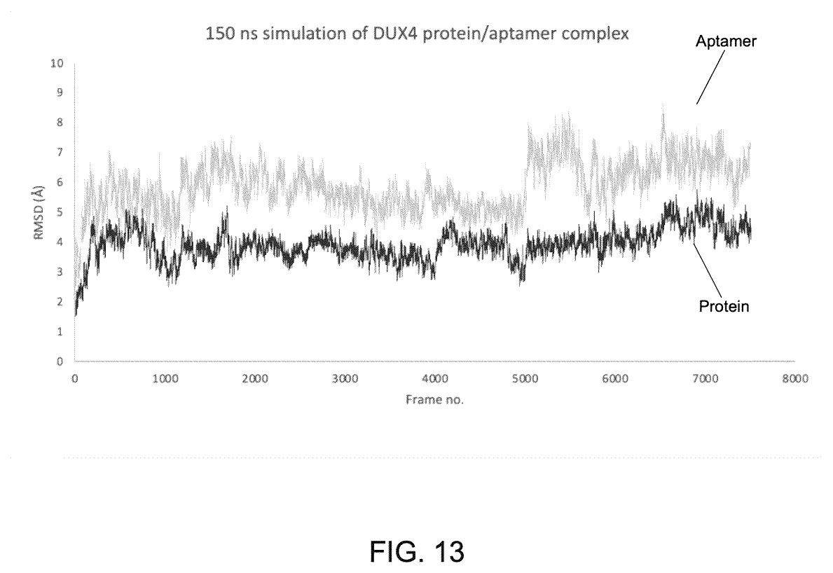

As further schematically illustrated in FIG. 21, it may be appreciated that if the principal angle α is provided by 180°, or, equivalently, by a value that is close to 180°, the oxygen atoms O3′ and O5′ of the terminal nucleotides of SAS1 and SAS2 may be connected using a rigid connector provided by double-stranded DNA helix without a junction. In this case, the same computational process as has been applied to determine the shape of a rigid connector with a junction (see FIGS. 2D-2H) may be used. Therefore, FIG. 2J may schematically illustrate a rigid connector without a junction, e.g., the connector C2, which may have been transformed (using translation and/or rotation operations) and extended to match the endpoints O1 and O2 of the connector C2. Furthermore, FIG. 2K may schematically illustrate the connector C2 as double-stranded DNA without a junction, which may be connected to the oxygen atoms O3′ and O5′ of the terminal nucleotides of SAS1 and SAS2 using single-stranded DNA, i.e., spacers D1′ and D2′, respectively.

FIG. 3 schematically illustrates global SAS assembly for a portion of an intended target molecule. It may be appreciated that the approximated surface of the target molecule is schematically illustrated by a smooth curve M, which corresponds to the contour of the cross-section the target molecule. In this example, a global SAS assembly may comprise four SASs, each containing four nucleotides schematically illustrated by white nodes, wherein SAS1 may be connected to SAS2 using a rigid connector with a junction C1′ (see FIGS. 2D-2H), SAS2 may be connected to SAS3 using a flexible connector C2′ (see FIGS. 2A-2C), and SAS3 may be connected to SAS4 using a rigid connector without a junction C3′ (see FIGS. 21-2K). It may be appreciated that the rigid connector C1′ may be connected to SAS1 and SAS2 using spacers D3 and D4, respectively, and the rigid connector C3′ may be connected to SAS3 and SAS4 using spacers D5 and D6, respectively.

Furthermore, in order to transform the global SAS assembly into a single, linear DNA sequence, a loop must be added at one of the two principal terminal nucleotides, which may be provided by the endpoints of global SAS assembly and, in this example, are denoted by PE1 and PE2. It may be appreciated by a person skilled in the art that while either endpoint PE1 or PE2 may be selected to create a loop, one endpoint may be preferable due to having more space to accommodate the loop structure. i.e. being less constrained. The next step may be to assess which one of the two principal terminal nucleotides PE1 or PE2 of the SAS global assembly may be less constrained. In order to perform this assessment, for each terminal oxygen atom of the principal terminal nucleotide of the global SAS assembly (e.g., the oxygen atom O5′ associated with the terminal nucleotide PE1 and located at the distance L1 from the surface M of an intended target molecule, and the oxygen atom O3′ associated with the terminal nucleotide PE2 and located at the distance L2 from the surface M of an intended target molecule), the closest atom of the target molecule may be identified. Therefore, the oxygen atom having the longest distance to the closest atom of the target molecule (for instance, see O3′ located at the distance L2 from M) may be less constrained and may be selected for placement of a loop. At the selected terminal nucleotide, in this example PE2, a flexible spacer (see spacer SP1) may be added to allow the sequence to turn back on itself. A spacer SP1 may connect to the connector C3′ on the opposite side to the single-stranded DNA spacer D6 connecting SAS4 to the connector C3′. In this example, the spacer SP1 may be constructed from single-stranded DNA and may consist of several thymine (T) nucleotides. However, a person skilled in the art may appreciate that the spacer SP1 may alternatively consist of any of A, C, G, or T nucleotides. The length of the spacer may be adjusted according to the length of DNA (SAS, spacers, simple connectors) to which it is opposite. For example, a spacer may be as short as four nucleotides, or may be longer, for example, containing eight or even ten nucleotides.

As further schematically illustrated in FIG. 3, to transform the global SAS assembly into a single, linear DNA sequence, flexible spacers, such as spacers SP2 and SP3, may be added between each double-stranded connector, i.e., in this example, SP2 may link the connectors C1′ and C3′ on the opposite side to the single-stranded spacers D4 and D5, respectively. In this example, the spacer SP2 may also be constructed from single-stranded DNA and may comprise a number of thymine nucleotides (or any other suitable nucleotides) to match the lengths of the SAS2 and SAS3, as well as spacers D4, D5 and simple connector C2′.

Once all SASs of a selected candidate SAS set have been connected using corresponding connectors, the global SAS assembly may provide the structure of an aptamer (for example, see a computationally synthesized aptamer illustrated in FIG. 8).

It may be appreciated that all atomic coordinate data (e.g., 3D-coordinates, angles, interatomic connections, etc.) associated with the global SAS assembly may be stored in PDB format. Furthermore, renumbering of the indices corresponding to the nucleotides constituting the global SAS assembly may be performed to facilitate further computations.

It may further be appreciated that once the structure of an aptamer has been obtained in silico using the above-described technique based on connecting SASs as schematically described in FIG. 1, a person skilled in the art may synthesize this structure in vitro and further assess the binding of an aptamer to an intended target molecule in a physical laboratory setting.

Furthermore, once the structure of an aptamer has been obtained using the process 100 schematically illustrated in FIG. 1, a potential candidate aptamer may be validated using the processes 200, 300 and 400 provided in FIGS. 5 to 7, respectively.

Furthermore, a person skilled in the art may appreciate that processes 200, 300 and 400 may be used for an aptamer that has been obtained by an alternative method to process 100 (see FIG. 1). It may be appreciated that an aptamer sequence may, for example, be determined via the SELEX method or selected from an existing database, such as, for example, SELEX database, Aptamer Target Database (ATDB), Aptamer Database, etc.

Once an aptamer has been computationally synthesized, it may be validated using the validation process 100′ schematically illustrated in FIG. 4. It may be appreciated that the validation process 100′ may help to increase certainty of successful simulation of binding between a computationally synthesized aptamer and an intended target molecule, which may reduce the rate of failure when aptamer is tested in the lab. For that, a computationally synthesized aptamer obtained using the method 100 shown in FIG. 1 may be represented as a sequence of nucleotides providing the 1D structure of an aptamer (see step 111 in FIG. 4). Then, the secondary (2D) structure of an aptamer may be predicted (see step 112 in FIG. 4). Various computational algorithms, which may typically include thermodynamic and energy minimization techniques, may be used for predicting the most stable secondary structure of the nucleic acid sequence. The algorithms for predicting 2D aptamer structures may include but are not limited to RNAfold, Mfold, NUPACK, and other suitable algorithms.

### Paragraph Removed

In this embodiment, the RNAfold algorithm, i.e., the algorithm that is a part of the ViennaRNA software, may be used to predict the minimum free energy (MFE) configuration of the 2D aptamer structure based on nearest-neighbor thermodynamics principles. The RNAfold algorithm may take as an input the data containing information about the aptamer sequence and output predicted MFE secondary aptamer structure. The output data may be stored in TXT format and may include information about base-pairing interactions, stem-loop structures, and overall folding topology of an aptamer. The resulting secondary (2D) structure of an aptamer may then be validated (see step 113 in FIG. 4).

It may be appreciated by a person skilled in the art that the validation process may involve but is not limited to assessing the stability of the 2D aptamer structure, comparing the predicted 2D structure to the structure synthesized in process 100 provided in FIG. 1, comparing the predicted 2D aptamer structure to existing secondary structures of similar aptamers (if such data is available), etc. The secondary aptamer structure may be stored, for example, in a computer-readable format, such as TXT format.

If the resulting 2D structure of an aptamer is not acceptable, i.e., for example, it is not consistent with the structure synthesized in process 100, the entire validation process 100′ may then terminate, and a new aptamer may be computationally synthesized using the process 100 provided in FIG. 1. It may be appreciated that a predicted secondary structure of an aptamer may be visualized using, for example, Visualization Applet for RNA (VARNA), which is a Java-based software tool for visualizing secondary RNA structures including aptamers, RNAplot, RNAstructure, and other suitable software.

As further schematically illustrated in FIG. 4 (see step 114), the tertiary (3D) aptamer structure (see, for example, FIG. 8) may be predicted. Various computational tools for predicting 3D nucleic acid structures, including aptamers, may include but are not limited to RNAComposer, RNAStructure, iFoldRNA software, etc. In this embodiment, the RNAComposer software may be used to predict and generate the model of the 3D structure of an aptamer using the information about the aptamer's primary and secondary structures. RNAComposer may employ a coarse-grained modeling technique, which represents aptamer's structure by grouping multiple atoms together to form pseudo-atoms (i.e., nucleotide clusters), such as, for example, groups of atoms that are found in spatial proximity and may exhibit similar physical and chemical properties. The 3D aptamer structure may be represented by RNA nucleotides, which involves specifying each nucleotide, and its coordinates, which define nucleotide spatial positions in the 3D space, and other chemical and physical properties.

Representing aptamer's 3D structure, which may comprise RNA nucleotides, by a group of clusters, instead of being represented by each individual atom, provides a major simplification for the computational modeling algorithm by offering reduced geometrical and computational complexity, reasonable computational time, faster sampling, as well as an easier way to explore various spatial structural configurations. The resulting predicted 3D RNA aptamer structure may be stored, for example, in PBD format or mmCIF (macromolecular Crystallographic Information File) format, which may include information about aptamer atom types, atomic coordinates, bond types, etc. The predicted 3D aptamer structure provided in RNA format may be visualized and further analyzed using molecular visualization software, including but not limited to PyMOL, VMD (Visual Molecular Dynamics), Jmol, UCSF Chimera, or any other suitable software.

In an alternative embodiment, it may be possible, using Al-based software, such as, for example, trRosettaRNA, to predict and construct 3D structure of an aptamer based only on the information about the aptamer's sequence.

As further illustrated in FIG. 4 (see step 115), in the case where a DNA aptamer is preferred, the predicted tertiary (3D) aptamer structure may be optionally converted from RNA to DNA. The conversion may be performed using PyMOL, BIOVIA, etc. In this embodiment, PyMOL may be used. During this process, each uracil (U) nucleotide in the predicted 3D RNA aptamer structure is replaced by the corresponding DNA nucleotide, i.e., thymine (T), and each ribose sugar is replaced by deoxyribose.

The resulting tertiary (3D) structure of an aptamer may then be validated (see step 116 in FIG. 4). If the resulting aptamer 3D structure is not acceptable, i.e., for example, it is not consistent with the structure synthesized in process 100, the entire validation process 100′ may then terminate, and a new aptamer may be computationally synthesized using the process 100 provided in FIG. 1.

It may be appreciated by a person skilled in the art that the process of aptamer structure validation 100′ may be used to predict a tertiary (3D) structure of an aptamer, whose 1D structure, i.e., nucleotide sequence, has been determined via the SELEX method or has been selected from an existing database, such as, for example, SELEX database, Aptamer Target Database (ATDB), Aptamer Database, etc. However, this approach may be not as nearly robust and may not always result in obtaining a desired 3D structure of an aptamer.

As illustrated in FIG. 5, once an aptamer's 3D structure has been validated, the process of docking an aptamer to an intended target molecule (see process 200) may begin with a preparation of separate water boxes (see step 210) for an aptamer and intended target molecule. It may be appreciated by a person skilled in the art that a water box may be provided by a cubic or parallelepiped cell comprising a specific number of water molecules, salt ions or other elements depending on the use case. Additionally, a separate water box may be prepared for potential docking of an aptamer to an intended target molecule (see step 240 of the process 200).

As illustrated in step 210 of FIG. 5, a computationally synthesized aptamer may be placed into a solution (see an aptamer, which is placed into a water box, schematically illustrated in FIGS. 9A and 9B), providing an environment that simulates an expected liquid medium for the target molecule detection, using molecular dynamics preparation software, such as, for example, OpenMM or AmberTools. In this embodiment, as an example, the OpenMM software may be used. This software includes a water box feature (i.e., in 3D, it may typically be provided by a cubic or parallelepiped cell, which may further comprise a specific number of water molecules, salt ions or other components depending on the use case), that may be prepared in order to provide an appropriate solution environment, allowing for realistic interactions between an aptamer and solvent molecules comprised within a solution, while ensuring that the entire system remains hydrated throughout the simulation process. Varying the number of solvent components comprised within a solution may have a direct impact on the atomic interaction sampling of the aptamer-solution system, and, therefore, this number may be varied to enhance the system's transitions between various states. Periodic boundary conditions may be applied to limit edge effects.

It may be appreciated by a person skilled in the art that the following parameters of a solution may also be specified: the lowest and highest possible values of potential of hydrogen (pH) level, which, for example, may be 7.4 for phosphate-buffered saline (PBS); salt ion types contained in a solution, such as positive ions (e.g., Cs⁺, K⁺, Li⁺, Na⁺, Rb⁺) and negative ions (e.g., Cl⁻, Br⁻, F⁻, I⁻); the lowest and highest possible values of salt concentration, which may be measured in units of molarity; the lowest and highest values of the temperature of a solution.

To simulate behavior of an aptamer in the solution, OpenMM may take as an input the 3D structure of the aptamer provided, for example, in PDB format, as well as the secondary input parameters, such as the lowest and highest pH and salt concentration levels of the solution, salt type, etc., and outputs the resulting 3D structure of an aptamer in the solvent environment, which may also be stored in PDB format.

Then, as further illustrated in FIG. 5 (see step 220), the 3D structure of an aptamer may be optimized to achieve its most stable configuration, or, equivalently, its minimum free energy (MFE) configuration (see FIG. 9B). In this embodiment, the OpenMM software may be used to tackle this task. Energy optimization of an aptamer-solution system may be achieved using numerical optimization algorithms, such as, for example, the conjugate gradient algorithm, line search algorithm or other suitable techniques, which may be included in the OpenMM software package, allowing for adjusting the spatial position of each atom, in order to minimize the total potential energy (TPE) of that system and achieve its minimum free energy MFE configuration. A force field, which includes bonded and non-bonded interactions between atoms, may be used to calculate the TPE of an aptamer-solution system. The energy minimization process may be iterative until some convergence criterion of an algorithm used to perform energy optimization is met. Next, the external parameters of the aptamer-solution system, such as for example temperature, pressure, volume, etc., may be applied and the system simulated using Langevin dynamics, Hamiltonian mechanics, or other molecular dynamics approaches, which may be included in OpenMM software package. The simulation may be performed under constant volume and temperature or constant pressure and temperature conditions for a period of 1 to 10 nanoseconds (ns), or until an aptamer-solution system equilibrium is achieved, i.e., that system has reached a state where its unconstrained properties are maintained over time. If the 3D aptamer structure may not be acceptable after energy optimization simulation, e.g., the optimization algorithm did not converge, etc., the simulation may be repeated with increased optimization time.

Once energy optimization step has been completed and an aptamer-solution system has reached its equilibrium configuration, the equilibrated 3D aptamer structure may be retrieved and stored, for example, in PDB format. The energy stability plot (not shown in the drawings), i.e., a plot comprising aptamer-solution energy values obtained over simulation time, may be also obtained as the result of the energy optimization process to validate that the resulting 3D aptamer structure has reached equilibrium. For the aptamer-solution system, to visualize the energy minimization simulation process, for example, PyMOL, AmberTools, or VMD (Visual Molecular Dynamics) software may be used, allowing for visualizing trajectories of atoms generated by atomic interactions and dynamics of aptamer-solution system, as well as molecular conformations. For example, the VMD software may also provide tools for calculating the system's properties, such as root-mean-square deviation (RMSD), root-mean-square fluctuation (RMSF), etc.

As further schematically illustrated in FIG. 5, if the structure of an intended target molecule has not yet been equilibrated in a solution as has been performed for an aptamer, the next step may be to prepare an intended target molecule for further binding with the computationally synthesized aptamer structure (see FIG. 1) in order to further validate the binding of the aptamer's structure to an intended target molecule (see step 200 in FIG. 5). In order to obtain an equilibrated 3D target structure, as an example, in this embodiment, the OpenMM software may be used. The 3D structure of an intended target molecule may be placed into a water box, which may contain the simulated expected liquid medium (see FIG. 10A). The number of solvent components comprised within a solution of the water box may be varied, to simulate a realistic solution environment.

In order to simulate behavior of an intended target molecule in the solution environment, the OpenMM software may take as an input the 3D structure of an target molecule provided, for example, in PDB format, as well as the secondary input parameters, such as, for example, the lowest and highest pH and salt concertation levels of the solution, salt type, etc., and outputs the resulting 3D target-solution system, which may also be stored in PDB format. Then, the 3D structure of an intended target molecule placed into a water box may be optimized to achieve its MFE configuration (see FIG. 10B schematically illustrating equilibrated 3D structure of an intended target molecule). In this embodiment, the OpenMM software may be used to perform energy optimization simulations. Energy optimization of the 3D target-solution system may be achieved using numerical optimization algorithms, such as, for example, the conjugate gradient algorithm, line search algorithm or other suitable techniques, which may be included in OpenMM software package, allowing for adjusting the spatial position of each atom, in order to minimize the TPE of that system and achieve its MFE configuration. A force field, which includes bonded and non-bonded interactions between atoms, may be used to calculate the TPE of a target-solution system. The energy minimization process may be iterative until some convergence criterion of an algorithm used to perform energy optimization is met. Next, the external parameters of the 3D target-solution system, such as for example temperature, pressure, volume, etc., may be applied and the system simulated using Langevin dynamics, Hamiltonian mechanics, or other molecular dynamics approaches, which may be included in OpenMM software package. The simulation may be performed under constant volume and temperature or constant pressure and temperature conditions for a period of 1 to 10 nanoseconds (ns), or until an aptamer-solution system equilibrium is achieved, i.e., that system has reached a state where its unconstrained properties are maintained over time. If the 3D target molecular structure may not be acceptable after energy optimization simulation, e.g., the optimization algorithm did not converge, etc., the simulation may be repeated with increased optimization time. Otherwise, the resulting equilibrated 3D target molecular structure may be stored, for example, in PDB format. Additionally, the energy stability plot may be also obtained as the result of the energy optimization process to validate that the resulting equilibrated 3D structure of an intended target molecule is in its equilibrium configuration and is ready for docking with an aptamer structure.

Once the 3D target molecular structure has been optimized, and it has achieved its equilibrium state, the preparation of docking of the computationally synthesized aptamer to the target molecule may begin (see step 240 in FIG. 5). In this embodiment, PyMOL or other software may be used to achieve this task. During the step of docking preparation, the equilibrated intended target molecule, as well as the equilibrated aptamer structure, may be removed from the water box.

As schematically illustrated in FIG. 5 (see step 250), the step of rigid molecular docking of the computationally synthesized aptamer structure to an intended target molecule may result in creating an aptamer-target complex. It may include computationally predicting the binding mode, affinity, and interactions between an aptamer and its target molecule. The molecular docking process may include but is not limited to binding site prediction, selecting a suitable docking algorithm depending on the structure of an aptamer and its target molecule, exploring docking configurations (i.e., calculating all possible molecular orientations and conformations in order to find more stable, energetically favorable configurations), analyzing all stable aptamer-target complexes, validating each predicted energetically favorable aptamer-target complex using experimental data (if such data is available).

To simulate molecular docking between an aptamer obtained using the process illustrated in FIG. 1 and an intended target molecule, a suitable software, such as, for example, AutoDock, DOCK, Glide, etc., may be used. In this embodiment, the AutoDock software may be used to predict molecular docking between the predicted equilibrated 3D aptamer structure and a target molecule. Both, the structure of an aptamer, and the structure of an intended target molecule may, for example, be provided in PDB format. The computationally synthesized aptamer structure may be passed to AutoDock as an input together with a molecular target structure, which may as well be equilibrated. The aptamer and target structures may be defined as entirely rigid, or some of their selected internal bonds may be defined as flexible.

As further illustrated in FIG. 5 (see step 250), during the step of molecular docking, an aptamer may interact with an intended target molecule, which may result in creating at least one docking pose of an aptamer-target complex. The number of docking attempts (further referred to as dockings) to perform between an aptamer and a target molecule may as well be specified as an input. A grid-based algorithm or any other suitable algorithm may be used to search for acceptable binding configurations between a target molecule and an aptamer. Additionally, for example, Lamarckian Genetic Algorithm (LGA) or any other suitable algorithm may be used to perform a local search of configurations of a target molecule (for example, see a few possible configurations illustrated in FIGS. 11A to 11C), allowing for more energetically favorable docking configurations. At the end of this step, a docking score may be calculated for each binding configuration of an aptamer-target complex, i.e., for at least one docking pose, for example, using but not limited to empirical scoring functions included in AutoDock software.

After the aptamer-target docking process has been completed, the resulting at least one docking pose of an aptamer may be stored, for example, in PDB format. After that, the resulting at least one docking pose of an aptamer may be validated, for example, using a docking score calculated during the docking process and/or experimental data (if such data is available). If the resulting aptamer-target docking pose or poses are not acceptable, i.e., for example, none of the poses align with the binding site selected as part of process 100 (see FIG. 1) or none of the corresponding docking scores are sufficient, the entire process starts over again by synthesizing a new aptamer sequence.

As further illustrated in FIG. 5 (see step 260), a 3D aptamer structure in a docked pose may be merged with a 3D target structure to produce a single molecular structure, (further referred to as a complex structure). In this embodiment, as an example, PyMOL software may be used as a tool for merging 3D docked aptamer-target complexes. The resulting merged 3D aptamer-target complex may be stored, for example, in PDB format.

Once the merged 3D aptamer-target complex has been obtained, there are two assessments that may be performed: binding and cross-reactivity assessment (see FIGS. 6 and 7).

As illustrated in FIG. 6, to further validate the obtained structure of a computationally synthesized aptamer, binding assessment 300 of a merged 3D aptamer-target complex may include evaluating the quality and stability of binding interaction between an aptamer and its target molecule. As further illustrated in FIG. 6, a merged 3D aptamer-target complex, the 3D aptamer structure in a docked position, and the 3D target structure in a docked position may be processed by a molecular dynamics simulation software, such as, for example, OpenMM or AmberTools. In this embodiment, as an example, the AmberTools software may be used. Processing the merged 3D aptamer-target complex, as well as the 3D aptamer structure in the docked position and the 3D target structure in the docked position, simultaneously may ensure consistent atom order and numbering.

As further illustrated in FIG. 6 (see step 310), the merged 3D aptamer-target complex is placed into a water box (e.g., in 3D, it may typically be provided by a cubic or parallelepiped cell, which may further comprise a specific number of water molecules, salt ions or other components depending on the use case), that may be prepared in order to provide an appropriate solvent environment, allowing for realistic interactions between an aptamer and solvent molecules comprised within a solution. Periodic boundary conditions may be applied to limit edge effects. The resulting solvated merged 3D aptamer-target complex, i.e., the 3D aptamer-target complex-solution system (see FIG. 12A), as well as the processed 3D aptamer-target complex, 3D aptamer, and 3D target systems, may be stored in a suitable file format (e.g., PRMTOP and/or INPCRD formats).

As further illustrated in FIG. 6 (see step 320), the molecular dynamics simulation is performed for the solvated merged 3D aptamer-target complex. In this embodiment, for example, the OpenMM software may be used for this task. The energy optimization of the solvated 3D merged aptamer-target complex may be performed, which may help to relax any steric clashes, correct bond lengths, adjust bond angles and minimize the TPE of the merged 3D aptamer-target complex-solution system using suitable optimization algorithms, such as, for example, conjugate gradient or steepest descent, allowing to iteratively minimize TPE until convergence is achieved. Next, the external parameters of the aptamer-solution system, such as for example temperature, pressure, volume, etc., may be applied and the system simulated using Langevin dynamics, Hamiltonian mechanics, or other molecular dynamics approaches, which may be included in OpenMM software package. Different constant states of the system, such as a constant number of atoms, volume, temperature (i.e., or NVT), as well as constant number of atoms, volume, and pressure (i.e., or NVP) and/or a constant number of atoms, pressure, and temperature (NPT) may be considered. Additional spatial and harmonic constraints may be applied to the merged 3D aptamer-target complex to prevent significant structural perturbations while allowing the solvent to equilibrate around it (see FIG. 12B).

The molecular dynamics simulations may be performed over extended intervals of time to obtain data about the conformational space occupied by the merged 3D aptamer-target complex, as well as record trajectory frames, which may provide the information throughout the simulation at regular time intervals about the positions, orientations, and velocities of all atoms constituting the complex. Trajectory frames may allow capturing the evolution of the merged 3D aptamer-target complex-solution system over time, while the merged 3D aptamer-target complex interacts with the solvent molecules and undergoes conformational and topological changes when exploring different binding modes. In this embodiment, the OpenMM software may be used to simulate molecular dynamics. The output of molecular dynamics simulations may be, for example, stored in Digital Crystallography Data (DCD) format, which may be provided by single precision binary FORTRAN files containing Cartesian coordinates of each atom, as well as its trajectories and velocities at various time points.

After the task of molecular dynamics simulation has been accomplished, the energy summary of the equilibrated merged 3D aptamer-target complex-solvent system may be stored, for example, in TXT format, as well as the trajectory frames of the equilibrated merged 3D aptamer-target complex may be, for example, stored in DCD or PDB formats. A suitable software, such as, for example, VMD, PyMOL, etc., which allows to visualize trajectories of all atoms that are comprised within the equilibrated merged 3D aptamer-target complex, may be used to visualize the trajectory frames generated by atomic interactions and dynamics, as well as molecular conformations. The trajectory frames may be stored, for example, in DCD (or X-PLOR) format, which are single precision binary FORTRAN files containing the information about the coordinates of each atom of the aptamer-target-solvent system in Cartesian coordinates at various time points.

As further illustrated in FIG. 6 (see step 330), the trajectory frames may be further transformed using, for example, AmberTools software, which takes merged 3D aptamer-target complex trajectories, as well as merged 3D aptamer-target complex-solution system, as an input. The trajectory transformations may be applied to a trajectory of a specific atom or a selected group of atoms of the merged 3D aptamer-target complex. The trajectory transformations may include but are not limited to translation by a specific vector, transformation by applying a transformation matrix, rotation around a specific axis or a specific angle, rescaling trajectory velocities, recentering a trajectory to remove periodic boundary artifacts, aligning each trajectory frame onto a reference structure, removing certain atoms from the trajectory, normalizing trajectories to ensure comparability between different trajectory frames, etc. The output containing the merged 3D aptamer-target complex trajectories may be stored in coordinate (CRD) format.

Trajectory frame analysis may be performed, for example, using AmberTools, VMD, MDtraj, or other suitable software. In this embodiment, for example, AmberTools software may be used. The file containing merged 3D aptamer-target complex trajectories, the file containing merged 3D aptamer-target complex-solution system information, and optionally, a key residue file containing a list of specific nucleic and/or amino acids, which may be included, for example, for alignment purposes, may be provided as input to AmberTools. Trajectory frames may be analyzed to extract relevant information about the binding interactions between the aptamer and the target molecule. The trajectories may be also downsampled to generate a set of uncorrelated frames for subsequent binding strength computations. The trajectory frames may be aligned using only protein backbone atoms, in order to calculate the aptamer's displacement from the protein. As a result, the output comprises transformed trajectory frames and may be stored in CRD format.

Furthermore, as provided in step 340 of FIG. 6, the stability of the merged 3D aptamer-target complex may be assessed by examining the displacement between individual atoms or groups of atoms comprised within the complex. In this embodiment, for this step, the AmberTools software may be used. The software may take as input the information about merged 3D aptamer-target complex-solution system and may output the root-mean-square deviation (RMSD), root-mean-square fluctuation (RMSF), hydrogen bonding patterns, solvent accessible surface area (SASA), etc., may be calculated for a particular input to provide the information about stability score of merged 3D aptamer-target complex. The output containing specific calculation results (e.g., RMSD for selected atoms) may be stored, for example, in TXT format. For example, FIG. 13 illustrates a plot depicting the root-mean-square deviation (RMSD) for each of 7500 trajectory frames. This plot may be interpreted as, for the first 5000 frames, the aptamer may not be moving away from its target. However, around the 5000th trajectory frame, a rapid increase of RMSD occurs, which may correspond to a structural rearrangement that may be examined visually by an expert to confirm whether the binding site has undergone a morphological shift. It may be appreciated by a person skilled in the art that any other suitable number of trajectory frames may be used to assess stability of the merged 3D aptamer-target complex.

As further illustrated in FIG. 6 (see step 350), binding strength calculations may be also performed. The binding strength calculations and affinity analysis, i.e., estimating binding free energy of the 3D aptamer-target complex, may be performed using suitable computational methods, which may include but are not limited to molecular dynamics simulations, free energy calculations, empirical scoring functions, etc. In this embodiment, for example, molecular dynamics simulation analysis by the AmberTools software may be used for this task. At this step, the files containing merged 3D aptamer-target trajectories, 3D aptamer structure, 3D target structure, merged 3D aptamer-target complex and merged 3D aptamer-target complex-solution system may be provided as input. Additionally, for the latter, the parameters of the solution (e.g., solvent type, pH level, salt concentration, temperature, etc.) may be provided. As well, as additional input, the binding strength estimation methods, which will be employed for the calculations, may be specified.

The binding strength estimation methods may comprise but are not limited to: molecular mechanics generalized Born surface area (MM-GBSA) and molecular mechanics generalized Poisson-Boltzmann surface area (MM-PBSA) methods, which decompose the total free energy of the merged aptamer-target complex into three terms, i.e., solvation effects, contributions of molecular mechanical interactions and entropy of the aptamer-target-solution system; free energy perturbation (FEP) methods, which are used to perturb the merged 3D aptamer-target complex-solution system from a bound state to unbound state to provide a change in entropy and in free energy; machine learning models, which are trained on experimental binding affinity data (if such models and data are available), may also be used to predict binding strengths. Additionally, to further characterize the merged 3D aptamer-target complex, per-residue energy decomposition, hydrogen bonding analysis and structural clustering may be performed to help estimating the binding strength between an aptamer and its target. The resulting binding strength estimation for a merged 3D aptamer-target complex may be stored, for example, in TXT format. The results obtained from binding strength estimation and affinity analysis may be validated using, for example, statistical methods and/or experimental data (if such data is available).

Once the binding strength estimation and affinity analysis have been performed and have met user requirements, the next step is to assess the cross-reactivity of an aptamer (see FIG. 7). Otherwise, the entire process may start from the beginning, i.e., from computationally synthesizing a new aptamer, as provided by method 100 shown in FIG. 1.

As schematically illustrated in FIG. 7, the cross-reactivity assessment 400 for a merged 3D aptamer-target complex evaluates the ability of an aptamer to specifically bind to its target molecule without interacting with other molecules. This process may start by first identifying at least one cross-reactive target molecule (see step 410). Overall, potential interfering target molecules or, equivalently, unintended target molecules, which may be present in the environment where the aptamer-target complex may be used. In this embodiment, for example, the unintended target molecules may have at least 60% structural similarity with the intended target molecule. However, any other unintended target molecules may be considered, such as proteins or small molecules at high abundance in the expected detection medium. All names of potential unintended target molecules among available cross-reactive target molecules may be stored, for example, in TXT format, as well as their structures may be provided in PDB format.

Then, each of the identified cross-relative target molecules may be tested. In this embodiment, for example, the OpenMM software may be used for this task. During this step, each potential candidate cross-reactive target molecule is tested separately, and may be placed in a water box (see step 420). OpenMM takes as input a 3D molecular structure of that cross-reactive target, which may be provided in PDB format, as well as the information about the solvent, such as, for example, the dimensions of a water box, the lowest and highest pH and salt concentration levels, solvent temperature, etc., and provides an output comprising cross-reactive 3D target-solution system, which may be provided in PDB format.

Then, the structure of the cross-reactive target molecule may be optimized to achieve its MFE configuration in the solvent, i.e., become equilibrated (see step 430). In this embodiment, the OpenMM software may be used for this task, as it allows for adjusting the spatial position of each atom to minimize the TPE of the cross-reactive 3D target-solution system. A force field, which includes bonded and non-bonded interactions between atoms, may be used to calculate the TPE of the cross-reactive 3D target-solution system. The energy optimization process may be iterative until some convergence criterion of an algorithm used to perform energy optimization is met and the cross-reactive 3D target-solution system's equilibrium is achieved. Next, the external parameters of the cross-reactive 3D target-solution system, such as, for example, temperature, pressure, volume, etc., may be applied and the system simulated using Langevin dynamics, Hamiltonian mechanics, or other molecular dynamics approaches, which may be included in OpenMM software package. The simulation may be performed under constant volume and temperature or constant pressure and temperature conditions for a period of 1 to 10 nanoseconds (ns), or until a cross-reactive 3D target-solution system equilibrium is achieved, i.e., that system has reached a state where its unconstrained properties are maintained over time. If the cross-reactive 3D target structure is not acceptable after energy optimization simulation, e.g., the optimization algorithm did not converge, etc., the simulation may be repeated with increased optimization time.

The resulting equilibrated cross-reactive target 3D molecular structure may be stored, for example, in PDB format. Additionally, the energy stability plot (not shown in the drawings) may be also obtained as the result of the energy minimization process to validate that the resulting equilibrated cross-reactive 3D target structure is in its MFE configuration and is ready for docking with an aptamer structure.

Once the equilibrated 3D structure of a cross-reactive target molecule is acceptable, the next step is to prepare it for docking (see step 440). This step may be performed using, for example, the PyMOL software. During this step, the equilibrated cross-reactive 3D target structure may be removed from the water box, and the output equilibrated cross-reactive 3D target-solution system may be stored, for example, in PDB format. Additional docking preparations may be achieved, for example, using PyMOL software, to visualize the 3D structure of equilibrated cross-reactive target molecule and make additional adjustments, for example, to its atomic spatial positions, bonds, angles, etc. The resulting 3D structure of a cross-reactive target molecule may be stored, for example, in PDB format.

As further schematically illustrated in FIG. 7 (see step 450), the simulation of molecular docking between an aptamer and a cross-reactive target molecule may be performed, during which a computationally synthesized aptamer (see FIG. 1) may interact with the selected unintended target. To tackle this task, suitable software, such as, for example, AutoDock, DOCK, Glide, etc., may be used. In this embodiment, the AutoDock software may be used to predict molecular docking between the predicted equilibrated cross-reactive target molecule and an aptamer. The equilibrated 3D structure of cross-reactive target molecule together with the equilibrated 3D structure of an aptamer obtained during the optimizing 3D aptamer-solution system (see step 220 in FIG. 5), may be passed to AutoDock as an input. The number of docking attempts between an aptamer and a cross-reactive 3D target structure may be also specified in the input. The aptamer and cross-reactive target structures may be defined as entirely rigid or select internal bonds may be defined as flexible. A grid-based algorithm or any other suitable algorithm may be used to search for acceptable binding configurations between the cross-reactive target molecule and the aptamer. Additionally, LGA or any other suitable algorithms may be used to perform a local search of configurations of the cross-reactive target molecule, allowing for more energetically favorable docking configurations.

Additionally, a binding score threshold, which specifies a maximum level of affinity between an aptamer and a cross-reactive target molecule, may help to distinguish between the desired binding of an aptamer with the target molecule and the undesired binding of an aptamer with a cross-reactive target molecule, i.e., when, for example, the structure of the unintended target has no similarity with the structure of the intended target. Such a threshold may be calculated and specified for a particular aptamer and a cross-reactive target molecule to ensure the absence of binding between them. If the resulting docked cross-reactive target molecule and aptamer complex may not be acceptable, i.e., the complex exceeds the binding threshold score, a new aptamer 1D structure may be defined, and the entire process starts from the beginning (see process 100 in FIG. 1). The resulting output of this step may comprise aptamer docking positions, which may be, for example, provided in PDB format, as well as docking scores, which may be provided, for example, in TXT format.

Once the binding and cross-reactivity assessments (see FIGS. 6 and 7) have been completed, the next step may be to chemically synthesize an aptamer.

1. A method of computationally synthesizing an aptamer, the method comprising:
acquiring a molecular-structure file encoding a target molecule;
defining a set containing a plurality of short nucleotide sequences, each one of said plurality of short nucleotide sequences comprising a plurality of three to six nucleotides;
simulating an interaction between each of the plurality of short nucleotide sequences and at least one binding site of the target molecule using a molecular-dynamics engine to determine at least one binding pose and a corresponding binding strength for each of the plurality of short nucleotide sequences, and retaining binding poses having the binding strength that respects a binding strength threshold;
determining a candidate subset containing at least two spatially non-overlapping short nucleotide sequences corresponding to retained binding poses, wherein adjacent terminal nucleotides of the at least two spatially non-overlapping short nucleotide sequences respect a distance threshold and are non-bondable to the target molecule;
defining at least one connector complementary to a binding site topology of the target molecule for connecting adjacent terminal nucleotides of neighboring nucleotide sequences of said candidate subset to define an initial three-dimensional structure of the aptamer, wherein said at least one connector maintains spatial orientation of said neighboring nucleotide sequences; and
generating and storing in the computer-readable medium the molecular-structure file containing the initial three-dimensional structure of the aptamer for chemically synthesizing the aptamer in vitro.

2. The method as defined in claim 1, further comprising:
inputting a one-dimensional structure of said aptamer, said one-dimensional structure corresponds to said initial three-dimensional structure of said aptamer and is provided by a sequence of nucleotides;
predicting a two-dimensional structure of said aptamer using said one-dimensional structure;
predicting a three-dimensional structure of said aptamer using said two-dimensional structure;
comparing said initial three-dimensional structure of said aptamer and a predicted three-dimensional structure of said aptamer; and
retaining said aptamer if said predicted three-dimensional structure closely matches corresponds to said initial three-dimensional structure.

3. The method as defined in claim 2, further comprising:
simulating an interaction between said aptamer and said target molecule in a liquid medium using a docking algorithm for predicting at least one docking pose of said aptamer and said target molecule, based on predicting a binding mode and affinity of said aptamer with said target molecule while maintaining said aptamer rigid to prevent said aptamer from bending during docking to said target molecule,
assessing binding between said aptamer and said target molecule by estimating said binding strength and a stability of said at least one docking pose while allowing said aptamer to conform to said target molecule in said at least one docking pose; and
validating said aptamer if said binding strength and said stability of said at least one docking pose respects a binding strength threshold.

4. The method as defined in claim 3, further comprising:
selecting at least one unintended target molecule having a similar structure to the target molecule and expected to be found in a test environment of said capable of interacting with said aptamer target molecule;
assessing cross-reactivity between said aptamer and said at least one unintended target molecule; and
retaining said aptamer if said binding strength, said stability of said at least one docking pose, and said cross-reactivity respect a predetermined threshold.

5. The method as defined in claim 4, wherein said at least one unintended target molecule has at least 60% of structural similarity with said target molecule.

6. (canceled)

7. The method as defined in claim 1, further comprising selecting a binding site, said binding site defining a region containing at least a portion of said target molecule.

8. The method as defined in claim 1, wherein said selecting of said target molecule further comprising placing said target molecule into said expected detection simulated liquid medium.

9. (canceled)

10. The method as defined in claim 1, wherein said at least one connector is connected to said neighboring nucleotide sequences using spacers, said spacers are provided by single strands of nucleotides.

11. The method as defined in claim 1, wherein said at least one connector is provided by a nucleic acid backbone.

12. The method as defined in claim 1, wherein said at least one connector is a flexible connector provided by a single strand of nucleotides, said single strand of nucleotides comprising at least one nucleotide.

13. The method as defined in claim 1, wherein said at least one connector is a rigid connector provided by two antiparallel complementary nucleotide strands forming a double helix.

14. The method as defined in claim 13, wherein said rigid connector has at least one junction, said at least one junction has a corresponding principal angle value from about 20° to 180°.

15. The method as defined in claim 13, further comprising connecting a less constrained end of said aptamer to one of said two antiparallel complementary nucleotide strands of said rigid connector using said single strand of nucleotides to form a loop.

16. The method as defined in claim 3, wherein said simulating an interaction between said aptamer and said target molecule results in forming at least one docked aptamer-target complex.

17. The method as defined in claim 16, wherein at least one energy optimization algorithm is used to minimize energy of said at least one docked aptamer-target complex placed in said expected detection simulated liquid medium, allowing to achieve molecular equilibrium of said at least one docked aptamer-target complex.

18. The method as defined in claim 17, further comprising:
calculating said binding strength between said aptamer and said target molecule of said at least one docked aptamer-target complex; and
creating at least one merged aptamer-target complex if said binding strength respects a binding strength threshold.

19. The method as defined in claim 17, wherein said calculating said binding strength further comprises simulating molecular dynamics between atoms of at least one docked aptamer-target complex and said expected detection simulated liquid medium to capture trajectory frames of said atoms of said at least one docked aptamer-target complex.

20. The method as defined in claim 3, wherein said simulated expected detection liquid medium is water, and wherein said target molecule is a protein.

21. A method of synthesizing an aptamer, the method comprising:
acquiring a protein file encoding a target protein molecule;
defining a set containing a plurality of short nucleotide sequences, each one of said plurality of short nucleotide sequences comprising three to six nucleotides;
simulating an interaction between each of the plurality of short nucleotide sequences and at least one binding site of the target protein molecule using a molecular-dynamics engine to determine at least one binding pose and a corresponding binding strength for each of the plurality of short nucleotide sequences, and retaining binding poses having the binding strength that respects a binding strength threshold;
determining a candidate subset containing at least two spatially non-overlapping short nucleotide sequences corresponding to retained binding poses, wherein adjacent terminal nucleotides of the at least two spatially non-overlapping short nucleotide sequences respect a distance threshold and are non-bondable to the target protein molecule;
defining at least one connector complementary to a binding site topology of the target protein molecule for connecting adjacent terminal nucleotides of neighboring nucleotide sequences of said candidate subset to define an initial three-dimensional structure of an aptamer, wherein said at least one connector maintains spatial orientation of said neighboring nucleotide sequences;
generating and storing in the computer-readable medium the molecular-structure file containing the initial three-dimensional structure of the aptamer; and
chemically synthesizing the aptamer in vitro based on a data contained in the molecular-structure file.
